(19) United States
(12) Patent Application Publication
(10) Pub. No.: US 2024/0277847 A1
(43) Pub. Date: Aug. 22, 2024

(54) BENCH-STABLE TRIAZENE COMPOSITIONS FOR PROTEIN MODIFICATION

(71) Applicant: ARIZONA BOARD OF REGENTS ON BEHALF OF THE UNIVERSITY OF ARIZONA, Tucson, AZ (US)

(72) Inventors: John C. Jewett, Tucson, AZ (US); Garrett J. Davis, Tucson, AZ (US)

(21) Appl. No.: 18/566,935

(22) PCT Filed: Jun. 6, 2022
(86) PCT No.: PCT/US2022/032371

§ 371 Date: Dec. 4, 2023

Related U.S. Application Data
(60) Provisional application No. 63/197,083, filed on Jun. 4, 2021.

(51) Int. Cl. A61K 41/00; A61K 47/54; A61K 49/00

(52) U.S. Cl. CPC A61K 41/0042; A61K 47/545; A61K 49/0052; A61K 49/0056

(57) ABSTRACT

Triazenes and methods of producing diazonium species from said traizenes using ultraviolet (UV) light, which provide a fast, easy, stable, scalable, and selectively-triggerable means of modifying aromatic nucleophiles, including those on protein surfaces. Thus, the present invention also includes triazenes for use as bioconjugates, e.g., for use in protein modification, for use as probes (including but not limited to detectable probes such as fluorescent probes), protein crosslinking, etc.

### CROSS-REFERENCES TO RELATED APPLICATIONS

This application claims benefit of U.S. Provisional Patent Application No. 63/197,083 filed Jun. 4, 2021, the specification(s) of which is/are incorporated herein in their entirety by reference.

### STATEMENT REGARDING FEDERALLY SPONSORED RESEARCH OR DEVELOPMENT

This invention was made with government support under Grant No. 1552568, awarded by National Science Foundation. The government has certain rights in the invention.

### BACKGROUND OF THE INVENTION

### Field of the Invention

The present invention relates to triazenes and aryl diazonium chemistry.

### Background Art

Aryl diazonium ions have long been known to react with various biological nucleophiles due to their high electrophilicity, most notably aromatic residues such as tyrosine, as well as histidine, and tryptophan. Though this reactivity incurs an advantage in selectivity, other challenges remain. Their electrophilicity makes them susceptible to hydrolysis. Additionally, in situ generation using concentrated acid is typically required. Not only does the necessity of harsh acidic conditions pose challenges to biocompatibility, the process consumes both time and materials. Though a select few aryl diazonium can be isolated as stable salts, their bench-life can be limited, and they remain prone to degradation due to temperature and moisture. Furthermore, storage of large amounts of diazonium salt can pose a danger due to their shock sensitivity and explosive potential.

### BRIEF SUMMARY OF THE INVENTION

The present invention describes triazenes and methods of producing diazonium species from said traizenes using ultraviolet (UV) light. The discovery that UV treatment of triazenes generated diazonium species was surprising given that others in the field would expect that treating triazenes with UV light would create radical species or that triazenes would need a very strong acid to produce a diazonium species.

This discovery provides a fast, easy, stable, scalable, and selectively-triggerable means of obtaining diazonium species via triazenes. The speed at which the triazene molecules can release the diazonium species, e.g., in seconds to minutes, can be extremely advantageous compared to other systems that have shown to take minutes to hours. This provides for an extremely rapid system for labeling molecules such as but not limited to proteins or materials with electron-rich aromatics. Furthermore, the molecules herein can be easily synthesized, stored, and used.

Without wishing to limit the present invention to any theory or mechanism, the molecules herein can be used for a variety of applications including but not limited to biological research and drug discovery applications. For example, the methods and compositions herein can be used for protein labeling applications, applications involving antibodies (e.g., for antibody drug conjugates), chimeras where two proteins are covalently linked, probes such as fluorescent probes (e.g., to track a protein), etc. The compositions herein may be immobilized onto surfaces/resins/solid supports, may be used to find protein reaction partners (e.g., other proteins, DNA, other molecules, etc.), etc. The compositions herein may also be used in protein-based materials where proteins will be linked together or to another surface (e.g., the triazene can be linked in either orientation). As discussed, the present invention is not limited to biological applications. The compositions herein may be used with materials where electron-rich aromatics can be linked to other materials via the chemistry described herein in the presence of light, e.g, UV light. In some embodiments, focused (or masked) light (in combination with compositions herein) is to fabricate materials.

Without wishing to limit the present invention to any theory or mechanism, it is believed that the compositions and methods of the present invention are advantageous because the compositions are easier to synthesize as compared to molecules such as triazabutadienes, which are nitrogen-containing diazonium precursors that can release a diazonium species in a pH-dependent manner. Further, the triazene-UV reactivity described herein provides an easy means for selectively producing the diazonium species.

The present invention also shows that triazenes can be synthesized and used to modify aromatic nucleophiles, including those on protein surfaces via treatment at low pH, or through UV initiated diazonium release. Furthermore, the present invention provides evidence that UV irradiation allows for protein modification via an isomerization mechanism. Because of the abundance of various piperidine analogs, as well as other secondary amines, the present invention allows for an expanded array of triazene compositions that may be used for a wide variety of applications.

The present invention also includes triazenes for use as bioconjugates, e.g., for use in protein modification, for use as probes (e.g., detectable probes such as fluorescent probes), crosslinking, or other applicable purposes. For example, the present invention also includes the modification of tyrosine residues, histidine residues, and tryptophan residues using diazonium molecules produced by triazenes reacted with UV light.

The present invention includes triazene molecules modified for a specific purpose. For example, in certain embodiments, the molecules are functionalized with orthogonal handles, e.g., alkynes, etc., or other molecules such as fluorophores (e.g., for imaging applications).

The present invention includes libraries of designed conjugates that may be stored safely for long periods of time and are readily accessible via multiple mechanisms of activation.

The present invention also provides methods of synthesis of the triazene compositions herein. The present invention also includes methods for producing an aryl diazonium (e.g., with the use of UV light), etc. using the triazene molecules herein.

As previously discussed, while the use of UV has been reported to have been used to perform photolysis of triazene compositions, it is believed that UV irradiation has not been previously shown to liberate diazonium from similar triazenes for the purpose of azo-coupling. The present invention describes new triazenes as well as triazenes that have been previously reported. Importantly, the present invention describes the use of triazenes in novel ways based on the surprising discovery that UV treatment of triazenes could generate diazonium species. For example, among many things, the present invention describes triazenes in the context of UV irradiation. The present invention also describes triazenes in the context of the production of azo-adducts, including those on protein surfaces.

The present invention features systems comprising a composition according to Formula A and UV light, wherein the UV light reacts with the triazene molecule to form a diazonium species. The present invention features systems comprising a composition according to Formula A and UV light, wherein the UV light reacts with the triazene molecule to form a diazonium species, wherein the first atom of R₂ and the first atom of R₃ are both an sp³ hybridized carbon, or R₂ and R₃ form a ring with N³ nitrogen as shown in Formula B and Formula C, e.g., R₂=CH₂—X—CH₂—CH₂—R₃ or CH₂—CH₂—X—CH₂—R₃ wherein Ra is C and X is CH₂, NH, S, or O; or R₃=CH₂—X—CH₂—CH₂—R₂ or CH₂—CH₂—X—CH₂—R₂ wherein R₂ is C and X is CH₂, NH, S, or O.

In some embodiments, the composition is according to Formula B or Formula C. The present invention features systems comprising a composition according to Formula A, Formula B, or Formula C; and UV light, wherein the UV light reacts with the triazene molecule to form a diazonium species, wherein in Formula A the first atom of R₂ and the first atom of R₃ are both an sp³ hybridized carbon, or R₂ and R₃ form a ring with N³ nitrogen as shown in Formula B and Formula C (e.g., R₂=CH₂—X—CH₂—CH₂—R₃ or CH₂—CH₂—X—CH₂—R₃ wherein R₃ is C and X is CH₂, NH, S, or O; or R₃=CH₂—X—CH₂—CH₂—R₂ or CH₂—CH₂—X—CH₂—R₂ wherein R₂ is C and X is CH₂, NH, S, or O). The present invention also features compositions comprising a triazene molecule according to Formula A, wherein the first atom of R₂ and the first atom of R₃ are both an sp³ hybridized carbon, or R₂ and R₃ form a ring with N³ nitrogen as shown in Formula B and Formula C (e.g., R₂=CH₂—X—CH₂—CH₂—R₃ or CH₂—CH₂—X—CH₂—R₃ wherein R₃ is C and X is CH₂, NH, S, or O; or R₃=CH₂—X—CH₂—CH₂—R₂ or CH₂—CH₂—X—CH₂—R₂ wherein R₂ is C and X is CH₂, NH, S, or O). The present invention also features a composition according to Formula A, Formula B, or Formula C, wherein R₁ is in the ortho, meta, or para position, wherein in Formula A the first atom of R₂ and the first atom of R₃ are both an sp³ hybridized carbon, or R₂ and R₃ form a ring with N³ nitrogen as shown in Formula B and Formula C (e.g., R₂=CH₂—X—CH₂—CH₂—R₃ or CH₂—CH₂—X—CH₂—R₃ wherein R₃ is C and X is CH₂, NH, S, or O; or R₃=CH₂—X—CH₂—CH₂—R₂ or CH₂—CH₂—X—CH₂—R₂ wherein R₂ is C and X is CH₂, NH, S, or O). The compositions are capable of releasing a diazonium species upon exposure to UV light.

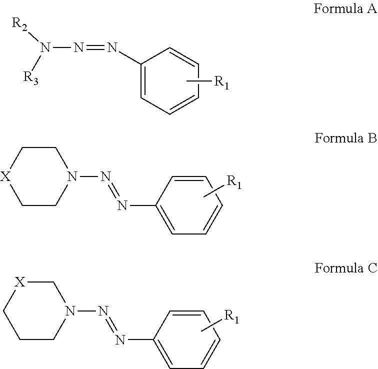

Referring to Formula A, Formula B, or Formula C: In some embodiments, R₁ is a carboxylic acid derivative. In some embodiments, R₁ is an alkyne. In some embodiments, R₁ is a bioorthogonal handle. In some embodiments, R₁ is a drug. In some embodiments, R₁ is selected from —NO₂, —CN, —CF₃, —COOH, —RCOOH, —CONHCH₃, —Br, —OMe, —H, or —CH₃. R₁ is not limited to the aforementioned examples; R₁ has the potential to be a wide variety of side groups, and one of ordinary skill in the art would understand mechanisms for customizing the triazene formulas herein with a wide variety of side groups.

Referring to Formula A, Formula B, or Formula C: In some embodiments, R₁ is in the ortho, meta, or para position. Referring to Formula B and Formula C (or Formula A if R₂=CH₂—X—CH₂—CH₂—R₃ or CH₂—CH₂—X—CH₂—R₃ wherein R₃ is C and X is CH₂, NH, S, or O; or R₃=CH₂—X—CH₂—CH₂—R₂ or CH₂—CH₂—X—CH₂—R₂ wherein R₂ is C and X is CH₂, NH, S, or O): In some embodiments, X is selected from CH₂, NH, S, or O. In some embodiments, X is an isotope. For example, an isotope may include but is not limited to an isotope of C or N, e.g., ¹³C, ¹⁵N, etc. of the Referring to the compositions and systems described above: In some embodiments, the compositions are water soluble. In some embodiments, the composition is stable at room temperature. In some embodiments, the composition is stable at room temperature for at least 6 months. In some embodiments, the composition is stable at room temperature for at least 1 year. In some embodiments, the composition is stable at room temperature for at least 5 years. In some embodiments, the composition is for protein modification. In some embodiments, the composition is a probe. In some embodiments, the composition comprises a detectable moiety or a tag. In some embodiments, the detectable moiety is a fluorescent moiety. In some embodiments, the detectable moiety is a colorimetric moiety. In some embodiments, the tag is an affinity tag. In some embodiments, the composition modifies aromatic nucleophiles. In some embodiments, the composition modifies tyrosine residues, histidine residue, tryptophan residues, or a combination thereof.

Referring to the aforementioned compositions and systems described above: In some embodiments, the UV light has a wavelength from 360-370 nm; in some embodiments, the UV light has a wavelength from 350-380 nm; in some embodiments, the UV light has a wavelength from 340-390 nm; in some embodiments, the UV light has a wavelength from 330-400 nm; in some embodiments, the UV light has a wavelength from 315-400 nm.

The present invention also features methods comprising exposing a composition according to the present invention to UV light, wherein the UV light causes the composition to release a diazonium species.

The present invention also features a method of producing a diazonium species. In some embodiments, the method comprises subjecting a triazene composition according to the present invention to UV light, wherein the UV light initiates diazonium release from the triazene composition.

The present invention also features a method of producing a triazene composition according to the present invention. In some embodiments, the method comprises conjugating a diazonium to a secondary amine. In some embodiments, the secondary amine is a proline residue. In some embodiments, the secondary amine is a lysine residue. In some embodiments, the lysine residue is a methylated lysine residue. In some embodiments, the secondary amine is a non-canonical amino acid. In some embodiments, the secondary amine is part of a peptide. In some embodiments, the secondary amine comprises piperidine or a derivative thereof. In some embodiments, the secondary amine comprises piperazine or a derivative thereof. In some embodiments, the secondary amine comprises morpholine or a derivative thereof. In some embodiments, the secondary amine comprises pyrrolidine or a derivative thereof. In some embodiments, the secondary amine comprises thiomorpholine or a derivative thereof. For reference, Nwajiboi et al. (Angew Chem Int Ed Engl, 2021, 60(13):7344-7352) describes triazenation reactions of secondary amines using arene diazonium salts to achieve tagging of monomethyl lysine in peptides.

The present invention also features a method of labeling a protein. In some embodiments, the method comprises introducing to the protein a composition according to the present invention and subjecting the protein to ultraviolet light, wherein the UV light initiates diazonium release from the triazene composition. In some embodiments, the diazonium reacts with tyrosine residues, histidine residues, tryptophan residues, or a combination thereof, of the protein. In some embodiments, the diazonium reacts with a non-canonical amino acid. In some embodiments, the labeling is covalent. In some embodiments, the labeling is reversible. In some embodiments, the labeling can be modified. In some embodiments, the labeling can be modified with a reducing agent. A non-limiting example includes sodium dithionite.

The present invention also features a method of labeling a protein with a detectable moiety. In some embodiments, the method comprises introducing to the protein a composition according to the present invention, wherein R1 comprises a detectable moiety; and subjecting the protein to ultraviolet light, wherein the UV light initiates diazonium release from the triazene composition, and the diazonium reacts with tyrosine residues, histidine residue, tryptophan residues, or a combination thereof, of the protein to bind the detectable moiety to the protein.

The present invention also features a method of labeling a protein with a detectable moiety. In some embodiments, the method comprises introducing to the protein a composition according to the present invention, wherein R1 comprises a detectable moiety; and subjecting the protein to ultraviolet light, wherein the UV light initiates diazonium release from the triazene composition, and the diazonium reacts with tyrosine residues, histidine residue, tryptophan residues, or a combination thereof, of the protein to bind the detectable moiety to the protein. In some embodiments, the method is for tracking the protein. In some embodiments, the method is for detecting the protein.

The present invention also features a method of crosslinking two molecules. In some embodiments, the method comprises introducing a first molecule to a second molecule, the first molecule having a triazene composition according to Formula A, Formula B, or Formula C incorporated therein and the second molecule having an electron rich aromatic group; and subjecting the molecules to ultraviolet light, wherein the UV light initiates diazonium release from the triazene composition of the first molecule, and the diazonium reacts with the electron rich aromatic group of the second molecule to crosslink the molecules. In some embodiments, the crosslink is covalent. In some embodiments, the crosslink is modifiable. In some embodiments, the first molecule, the second molecule, or both the first and second molecules are proteins.

The present invention also features a method of detecting a reaction partner with a protein, the protein having a composition according to the present invention incorporated therein. In some embodiments, the method comprises subjecting the protein and one or more possible reaction partners to ultraviolet light, wherein the UV light initiates diazonium release from the triazene composition, and the diazonium reacts with a binding partner having an electron rich aromatic group. Compositions and/or methods herein, such as the aforementioned method, may feature a triazene composition wherein R1 features a protein (or amino acid) and a detectable moiety, and thus R1 in diazonium form reacts with the target, e.g., reaction partner, e.g., electron rich aromatic group. In some embodiments, the detectable moiety is a tag. In some embodiments, the detectable moiety is a label.

The present invention also features a method of synthesizing an antibody-drug conjugate. In some embodiments, the method comprises introducing a drug to an antibody, the drug being R1 of a triazene composition according to the present invention, and subjecting the drug and antibody to UV light, wherein the UV light initiates diazonium release from the triazene composition, and the diazonium reacts with the antibody to bind the drug to the antibody.

The present invention also features a method of immobilizing a composition (the composition being R1 of a triazene composition according to the present invention) on a surface having electron rich aromatic groups. In some embodiments, the method comprises introducing the composition to the surface and subjecting the surface and the triazene composition to UV light, wherein the UV light initiates diazonium release from the triazene composition, and the diazonium reacts with the electron rich aromatic groups of the surface to bind the composition to the surface. In some embodiments, the surface is a resin or solid support.

The present invention also features methods of labeling a molecule. In some embodiments, the method comprises introducing to the molecule a triazene composition according to the present invention and subjecting the molecule to ultraviolet light, wherein the UV light initiates diazonium release from the triazene composition, and the diazonium reacts with the molecule to label the molecule. In some embodiments, the labeling is covalent. In some embodiments, the labeling is reversible or modifiable. In some embodiments, the molecule is a protein.

Referring to the aforementioned methods: In some embodiments, the UV light has a wavelength from 360-370 nm; in some embodiments, the UV light has a wavelength from 350-380 nm; in some embodiments, the UV light has a wavelength from 340-390 nm; in some embodiments, the UV light has a wavelength from 330-400 nm; in some embodiments, the UV light has a wavelength from 315-400 nm.

The present invention also features a kit comprising a triazene composition according to the present invention, e.g., according to Formula A, Formula B, or Formula C, and a set of instructions or access thereto that provides a method for producing a diazonium species from said composition.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a scheme of phenyl diazenyl piperidine triggered release of aryl diazonium.

FIG. 2 shows a scheme of synthesis of phenyl diazenyl piperidine triazene.

FIG. 3A shows diazonium reactivity with resorcinol can be triggered via release within the presence of pH 4 media or irradiation with 370 nm UV at pH 7. Samples were incubated at pH 4, or at pH 7 and irradiated for 3 hr prior to being extracted with CH₂Cl₂ (DCM).

FIG. 3B shows the percent yields of azo-adducts of triazenes (2a-i) with varying substituent electronics in the presence of pH 4 citrate buffer and via 370 nm UV at pH 7.

FIG. 3C shows formulas for compositions according to the present invention.

FIG. 4 shows a comparison on the effect of ring structure and size on relative diazonium release via treatment of various triazenes with 1 hr 370 nm UV irradiation at pH 7.

FIG. 5 shows a scheme of UV driven isomerization of the triazene scaffold promoting protonation at the N³ position and leading to diazonium release.

FIG. 6 shows a general reaction scheme for 1h irradiated in the presence of resorcinol.

FIG. 7 shows a general mechanism for triazenes pre-acidified with HCl (˜pH 1) and then added to protein solution for modification.

FIG. 8 shows a general reaction scheme for protein labeling by 6a following UV irradiation. Conjugation of AlexaFluor azide (488 nm) was done using standard copper click conditions with the addition of THPTA ligand.

FIG. 9 shows a comparison of reactivities, targets, and other features of traizene compositions of the present invention, as compared to triazabutadiene molecules and MaMa molecules previously described.

FIG. 10A shows a non-limiting example of a dimeric triazene, e.g., a homodimeric triazene.

FIG. 10B shows a non-limiting example of a dimeric triazene, e.g., a heterodimeric triazene.

FIG. 10C shows a non-limiting example of a water soluble monomeric piperazine triazene.

FIG. 11 shows a schematic of the design and concept for a hetero-dimeric pro-fluorophore construct capable of fluorescently labeling proteins.

FIG. 12 shows a reaction scheme for the NMR Kinetics experiment using 1 equiv. of 1h and 1 equiv. of resorcinol in deuterated methanol.

FIG. 13 shows the treatment of ethynyl triazenes 6a-b with cresol at pH 7 in the presence of 370 nm irradiation.

FIG. 14 shows non-limiting examples of compositions described in the present invention.

FIG. 15 shows non-limiting examples of piperidine triazenes.

### DETAILED DESCRIPTION OF THE INVENTION

The present invention describes triazenes and methods of producing diazonium species from said traizenes using ultraviolet (UV) light. The discovery that UV treatment of triazenes generated diazonium species was surprising given that others in the field would expect that treating triazenes with UV light would create radical species or that triazenes would need a very strong acid to produce a diazonium species. This discovery provides a fast, easy, stable, scalable, and selectively-triggerable means of obtaining diazonium species via triazenes. Without wishing to limit the present invention to any theory or mechanism, it is believed that the compositions and methods of the present invention are advantageous because the compositions are easier to synthesize as compared to molecules such as triazaubtadienes, and the UV reactivity provides an easy means for selectively producing the diazonium species.

The present invention also shows that triazenes can be easily synthesized and used to modify aromatic nucleophiles, including those on protein surfaces via treatment at low pH, or through UV initiated diazonium release. Furthermore, the present invention provides evidence that UV irradiation allows for protein modification via an isomerization mechanism. Because of the abundance of various piperidine analogs, as well as other secondary amines, the present invention allows for an expanded array of triazene compositions that may be used for a wide variety of applications.

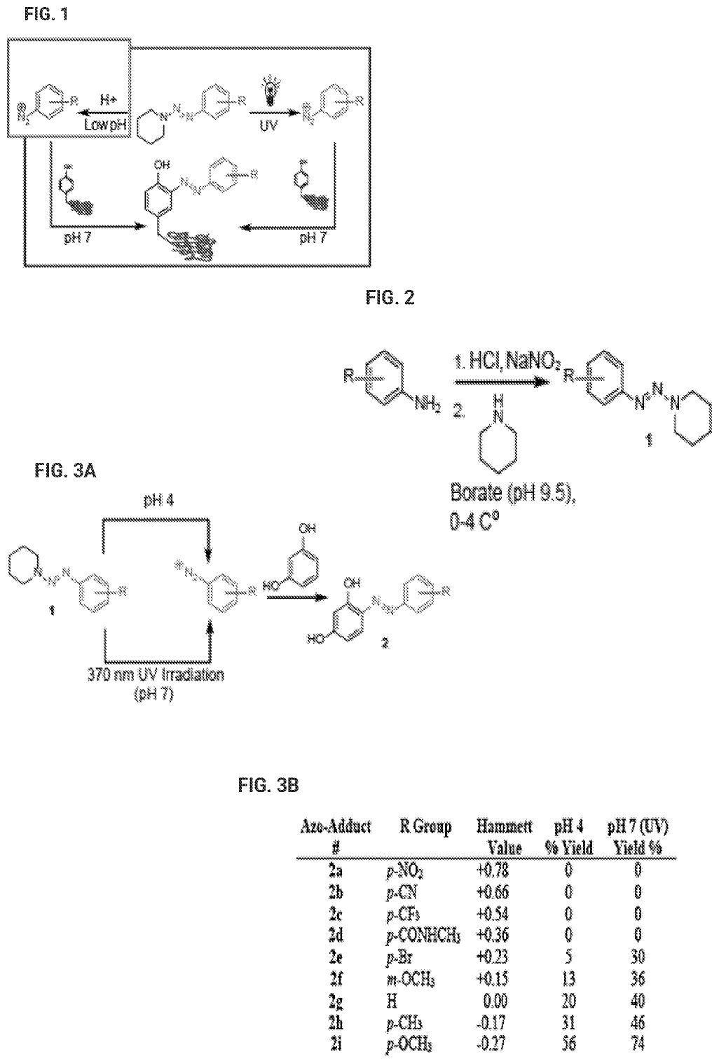

The present invention features triazene compositions synthesized by diazonium conjugation to secondary amines functioning as masked aryl diazonium molecules. Non-limiting examples of secondary amines include piperidine, piperazine, morpholine, and pyrrolidine, thiomorpholine, other piperidine-like molecules, etc.). The triazene compositions herein are bench stable. The compositions herein can release the aryl diazonium upon irradiation with UV light, as well when subjected to acidic conditions (see FIG. 1).

The present invention provides methods of use of the triazene compositions herein. The triazene compositions herein may be used for a variety of applications including but not limited to bioconjugation applications. In certain embodiments, the compositions are used for protein crosslinking (e.g., homodimeric versions with alkyne handles). In certain embodiments, the compositions are used for protein functionalization. In certain embodiments, the compositions are used for reversible functionalization. In certain embodiments, the compositions are used for crosslinking, e.g., via UV irradiation, acid treatment, etc. In certain embodiments, the compositions are used for protein purification. In certain embodiments, the compositions are used for imaging, e.g., for labeling. In certain embodiments, the compositions are used for protein capture, e.g., pull-downs. In certain embodiments, the compositions can be used on solid supports. The present invention is not limited to the aforementioned applications.

The molecules can be functionalized for various purposes. For example, in certain embodiments, the molecules are functionalized with orthogonal handles, e.g., alkynes, etc., or other molecules such as fluorophores (e.g., for imaging applications). Further, the present invention provides the ability to synthesize pro-fluorophore systems or fluorogenic scaffolds.

Referring to FIG. 2, triazenes may be synthesized using anilines. Bulk aniline starting materials were treated with standard diazonium conditions using sodium nitrite and HCl. Following diazotization, the solution was added to excess piperidine in an alkaline borate solution (˜pH 9.5). The resulting precipitate allowed for filtration of pure triazene products (1a-i). It was found that anilines with more positive Hammett values attributed to their aryl substituents afforded higher yields of respective triazene, consistent with the expectation of increased electrophilicity of the diazonium.

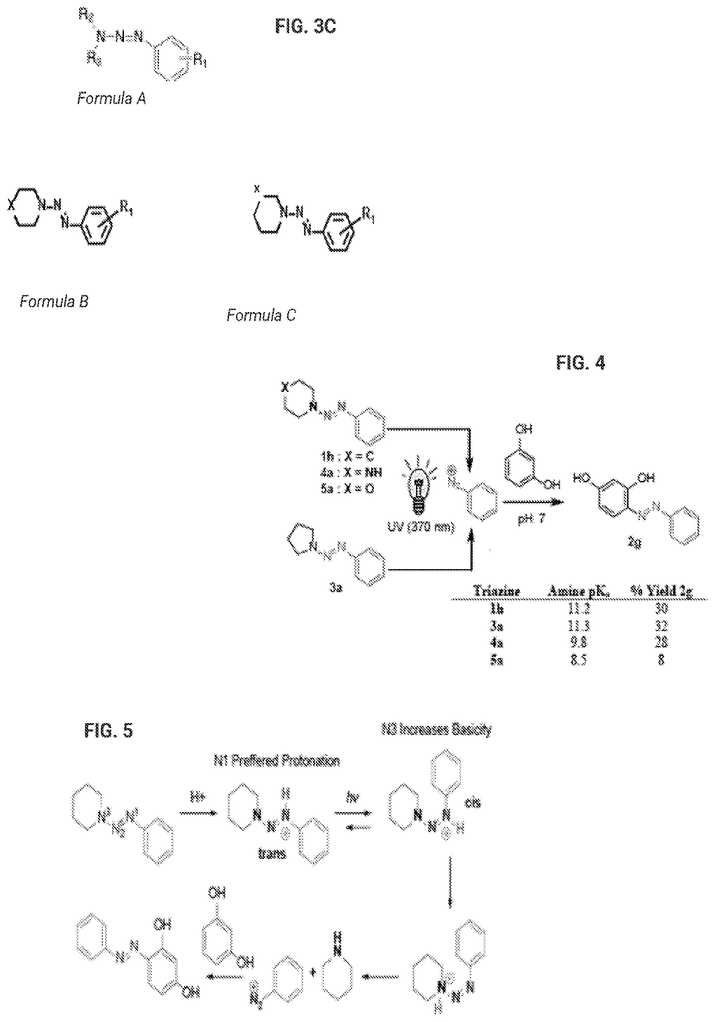

FIG. 3A shows a simple benzene scaffold (1g) was challenged with resorcinol in mildly acidic conditions (0.1 M pH 4 citrate buffer). Treatment of 1g with 1 equivalent of resorcinol in pH 4 citrate buffer yielded the respective azo-adduct (2g) after 3 hours (20%), showing that diazonium could be released in mildly acidic conditions (see FIG. 3B). Other analogs were challenged to determine whether electronics of the substituent could dictate reactivity. Indeed, it was found that the highly donating analogs such as p-methoxy (1i) and p-methyl (1h) triazene produced higher yields of azo-adduct (56% and 31% respectively) than 1g, while the more withdrawing substituents such as m-OCH₃ and p-Br had diminished yields. Interestingly, the p-nitro (1a), p-nitrile (1b), p-CF₃ (1c) and the p-CONHCH₃ analogs failed to produce any of the corresponding azo-adducts (see FIG. 3B).

The present invention is not limited to the specific compositions shown or described herein and includes numerous triazenes, such as those according to the formulas in FIG. 3C.

As shown in FIG. 4, a 370 nm UV LED light was shown to liberate the aryl diazonium. Diazonium liberation could be tracked via color change of the solution and TLC. After 3 hours of irradiation, products were extracted with CH₂Cl₂ (DCM) and analyzed via crude NMR. The resulting azo-adducts, if present, were purified on silica. It was found that diazonium coupling was increased amongst triazenes with lower aryl substituent Hammett Values. The highest amount of azo-adducts formed from the p-OMe (1i, σ=−0.27) and p-methyl (1h, σ=−0.17) analogs respectively.

Several different benzene triazenes were made to determine if they could all undergo UV initiated diazonium release. Using pyrrolidine, piperazine, and morpholine, respective benzene triazenes were synthesized (3a, 4a, 5a respectively). Alongside 1h, all were treated with 1 equivalent of resorcinol within a 50% mixture of MeOH and 0.1 M PBS buffer (pH 7) and irradiated for 1 hr (see FIG. 4). Following purification of the azo-adduct 1h, relative yields suggested that the piperidine, pyrrolidine and piperazine analogs all had similar reactivities, producing 30%, 32%, and 28% yields respectively. The morpholine derivative had a yield of 8%.

Referring to FIG. 5, 0.5 mM of triazenes 1a, 1g, and 1i were incubated in a water solution and the relative pH was measured before, during, and after irradiation with 370 nm light. Irradiation of the p-NO₂ analog (1a) for 1 minute did not elicit notable pH change. However, irradiation of the benzene analog (1g) and the p-OMe analog (2i) both illustrated notable increases of pH during the time of irradiation. Importantly, the relative change in pH observed increased with more donating substituents, whereas the change observed for 1i was over double that of 1g. Furthermore, the data notes that after irradiation has ceased, there is an immediate drop in pH consistent with the reversion of the scaffold to the trans-species. Additionally, the pH does not drop to nascent levels, which is also consistent with the release of piperidine following protonation at the N3, and thereby increasing the overall pH of the solution due to piperidine's high pKa.

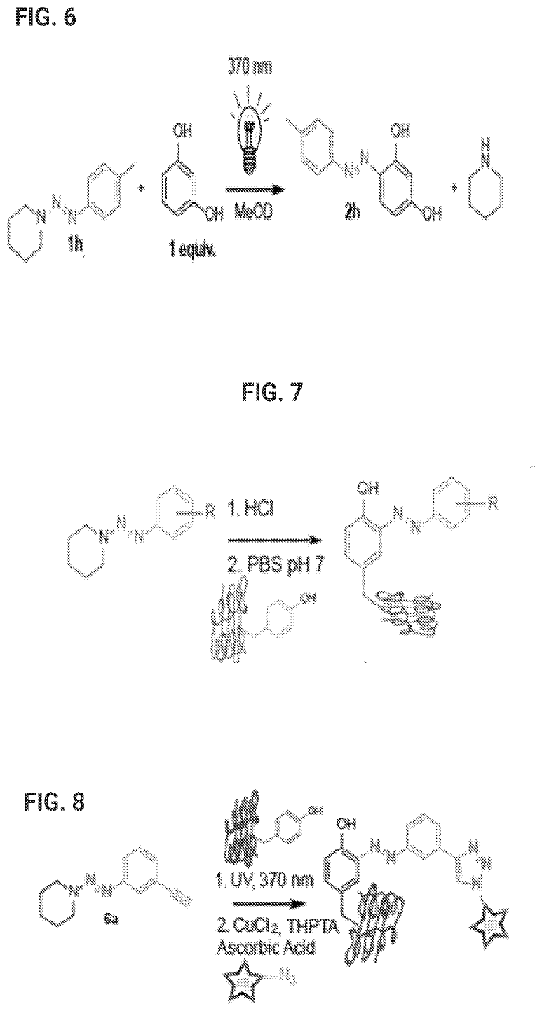

FIG. 6 shows a general reaction scheme for 1 h irradiated in the presence of resorcinol. FIG. 7 shows a general mechanism for triazenes pre-acidified with HCl (˜pH 1) and then added to protein solution for modification. FIG. 8 shows a general reaction scheme for protein labeling by 6a following UV irradiation. Conjugation of AlexaFluor azide (488 nm) was done using standard copper click conditions with the addition of THPTA ligand.

The present invention shows that these triazenes can be easily synthesized and used to modify aromatic nucleophiles, including those on protein surfaces via treatment at low pH, or through UV initiated diazonium release. Furthermore, the present invention provides evidence that UV irradiation allows for protein modification via an isomerization mechanism. Because of the abundance of various piperidine analogs, as well as other secondary amines, the present invention provides an expanded array of triazene compositions that may be used for a wide variety of applications.

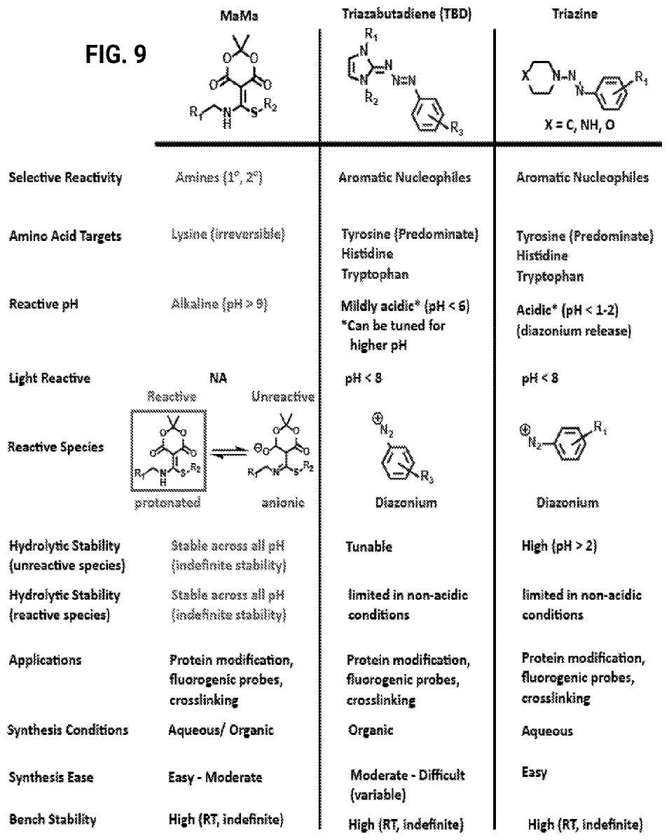

FIG. 9 shows a comparison of reactivities, targets, and other features of traizene compositions of the present invention, as compared to triazabutadiene molecules and MaMa molecules previously described. Without wishing to limit the present invention to any theory or mechanism, it is believed that the compositions and methods of the present invention are advantageous because the compositions are easier to synthesize as compared to molecules such as triazaubtadienes, and the UV reactivity provides an easy means for selectively producing the diazonium species.

The present invention also describes triazenes derived from cyclic amines for photo-initiated diazonium release and protein labeling. For example, the present invention describes dimeric triazenes. Homodimeric triazenes containing multiple protected diazonium species can be easily synthesized via treatment of piperazine with 2 or more equivalents of diazonium in aqueous alkaline conditions (pH >9). Although the monomeric species also forms, the dimeric species is preferentially made and readily crashes out of aqueous solution and therefore is easily purified by gravity filtration.

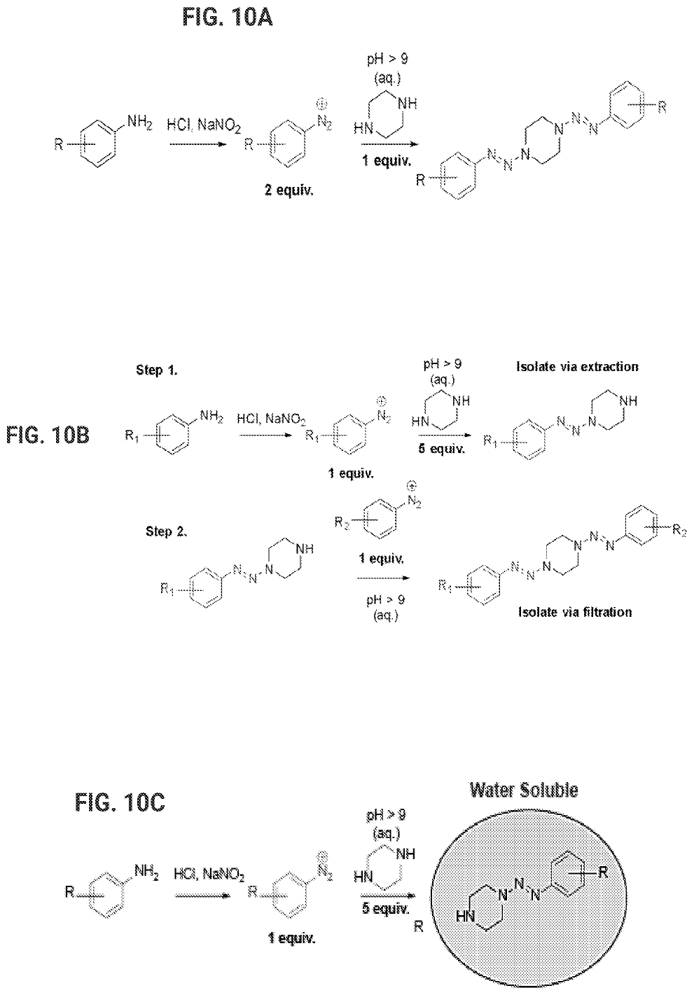

Referring to FIG. 10A, FIG. 10B, and FIG. 10C, heterodimeric triazenes containing two different protected diazonium species can be easily synthesized via a two-step process. In the first step, treatment of the desired diazonium species with excess piperazine will favor the production of the monomeric species. The monomer can be isolated by aqueous extraction using CH₂Cl₂ (DCM). After isolating the monomer, it can be reconstituted in buffer (pH >9) and treated with a second diazonium species of choice in 1:1 equivalency. The second conjugation will cause the product to precipitate and be easily isolated via gravity filtration. The present invention also describes water soluble monomeric piperazine triazenes. Monomeric protected diazonium species can be made via treatment of the desired diazonium species with excess piperazine at pH 9. This will favor the production of the monomeric species, which is water soluble. The monomer can be isolated by aqueous extraction using CH₂Cl₂ (DCM).

While other variations are water insoluble, the monomeric species have the benefit of being water soluble and therefore not requiring additional organic solvents to be made soluble in aqueous solutions. This allows for treatment of proteins without addition of organic solvents, and thereby allowing for a more biologically friendly environment. This is increasingly important for more complex biological studies including treatments of cells, larvae, or other whole organisms.

As previously discussed, the molecules herein can be functionalized for various purposes. For example, in certain embodiments, the molecules are functionalized with orthogonal handles, e.g., alkynes, etc., or other molecules such as fluorophores (e.g., for imaging applications). Further, the present invention provides the ability to synthesize pro-fluorophore systems or fluorogenic scaffolds.

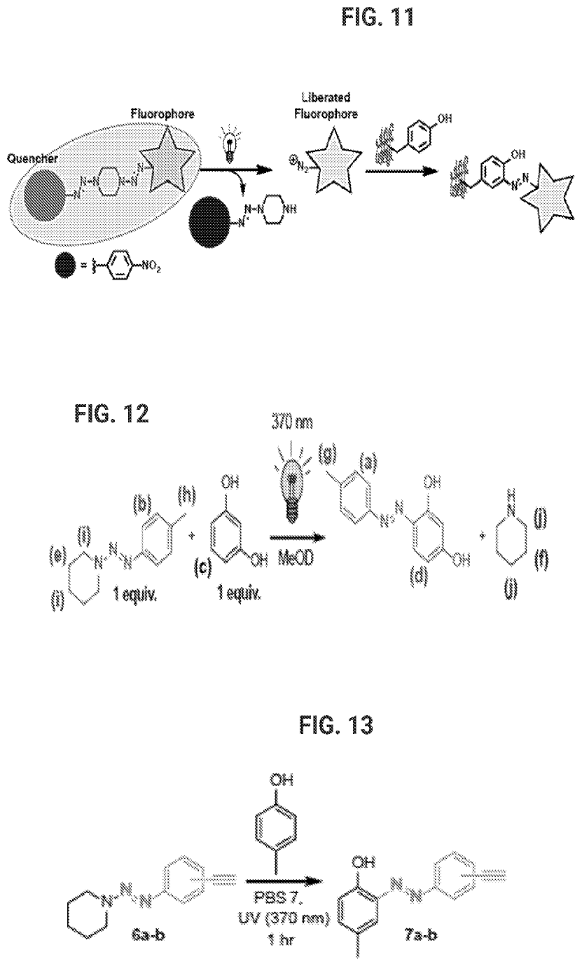

As shown in FIG. 11, as an example, a pro-fluorophore system may be fashioned from a piperazine scaffold via the synthesis of a hetero dimeric triazene. Coupling of a combination of a quencher diazonium on one side and a fluorescent diazonium on the other may elicit a non-fluorescent molecular that upon liberation of the fluorescent diazonium by UV irradiation or other means, would result in a diazonium capable of fluorescently labeling proteins. The present invention is not limited to the example in FIG. 11.

### EXAMPLE 1: 1-(Phenyldiazenyl)Piperidine Scaffold for Development of Protected Diazonium Capable of Initiated Release and Protein Labeling

The following is a non-limiting example of the present invention. It is to be understood that said example is not intended to limit the present invention in any way. Equivalents or substitutes are within the scope of the present invention.

In a 20 mL glass synthetic vial, aniline (1 eq.) was measured and subsequently placed on an ice bath (0-4 C). 4 mL of 10% HCl solution in water was added to the reaction vessels and magnetically stirred until the solution is clear and the aniline is completely dissolved. In a 1.6 mL Eppendorf tube, sodium nitrite (5 eq.) was measured out and dissolved in 1 mL of nanopore water and placed on ice. Once cool, the sodium nitrite solution is added slowly over 5-10 minutes, typically noting a distinct color change from yellow-orange solution to clear. Diazotization reactions are stirred for 30 minutes. In a separate 50 mL Erlenmeyer flask, piperidine, or pyrrolidine (5 eq) is added to 20 ml of 0.2 M Borate buffer pH 9.5 and stirred on ice. After 30 minutes, diazonium is slowly added to the alkaline piperidine solution, in 0.5 mL additions, noting color change and precipitation upon addition. Reaction pH is monitored by pH paper following each 0.5 mL addition. 10% NaOH is added as needed to keep the reaction solution basic. Upon final addition, the reaction is stirred for 1 hour on ice. The reaction solution is then neutralized with addition of HCl, causing increased precipitation. Precipitate was then isolated by gravity filtration and washed with nanopore water to remove excess piperidine, or pyrrolidine. Extraction with organic solvents (MeOH, acetone, or DCM) should be performed to remove excess salts. Products were characterized by NMR using CDCl₃.

FIG. 13 shows the treatment of ethynyl triazenes 6a-b with cresol at pH 7 in the presence of 370 nm irradiation for 1 hour yielded 33% (7a) and 31% (7b) of the desired cresol-ethynyl azo-adducts respectively.

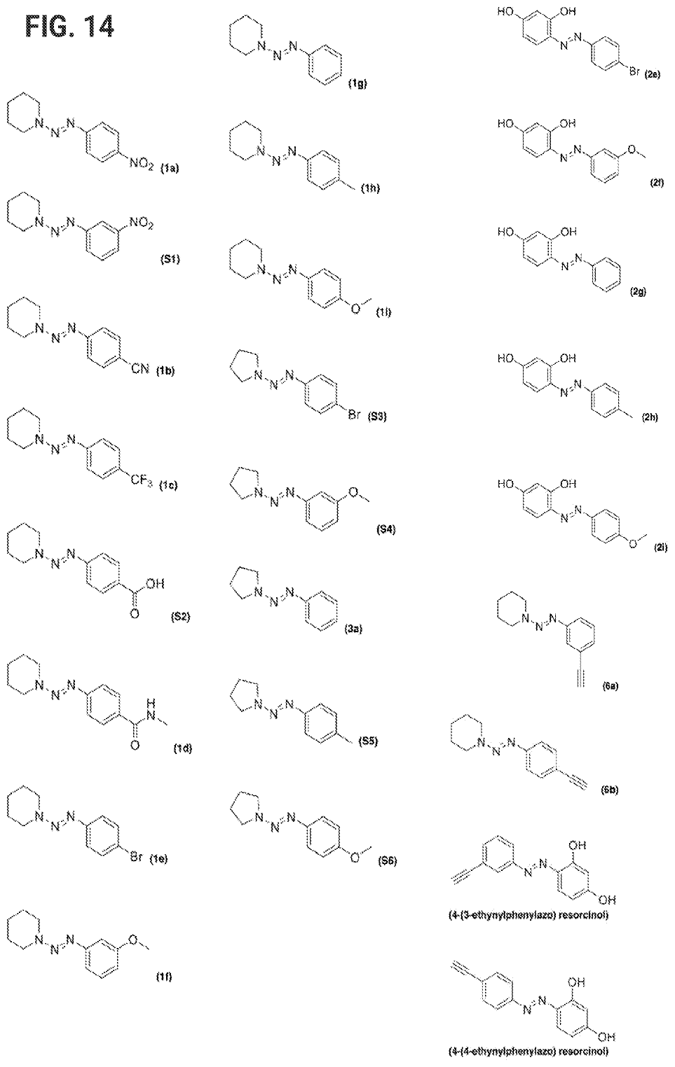

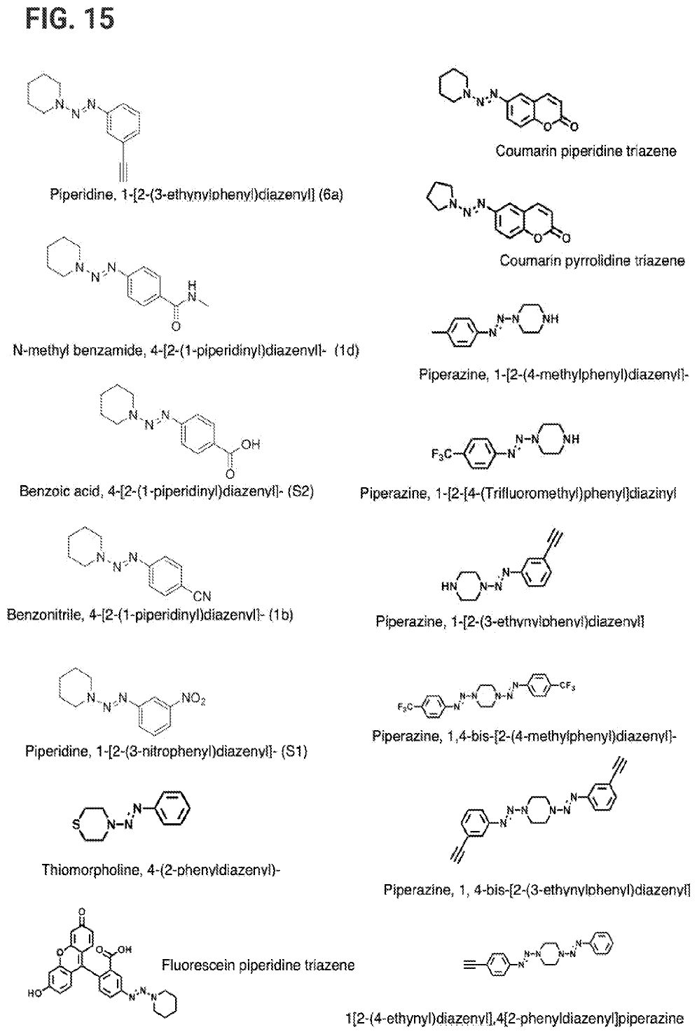

FIG. 14 and FIG. 15 show non-limiting examples of structures of the present invention. The present invention also includes triazene with various secondary amines. The present invention also includes triazenes for fluorescent labeling of proteins. The present invention also features piperazine homodimers.

Embodiments of the present invention can be freely combined with each other if they are not mutually exclusive.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A system comprising:
(a) a composition comprising a triazene molecule according to Formula A; and
(b) UV light; wherein the UV light reacts with the triazene molecule to form a diazonium species.

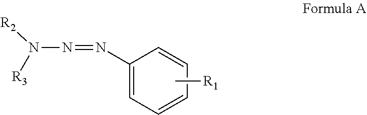

2. The system of claim 1, wherein the first atom of R₂ and the first atom of R₃ are both an sp³ hybridized carbon, wherein R₂=CH₂—X—CH₂—CH₂—R or CH₂—CH₂—X—CH₂—R₃, wherein R₃ is C and X is CH₂, NH, S, or O; or R₃=CH₂—X—CH₂—CH₂—R₂ or CH₂—CH₂—X—CH₂—R₂, wherein R₂ is C and X is CH₂, NH, S, or Q.

3. (canceled)

4. The system of claim 1, wherein R₁ is a carboxylic acid derivative, is an alkyne, is a bioorthogonal handle, or is a drug.

5.-7. (canceled)

8. The system of claim 1, wherein R₁ is selected from —NO₂, —CN, —CF₃, —COOH, —RCOOH, —CONHCH₃, —Br, —OMe, —H, or —CH₃.

9. The system of claim 1, wherein R₁ is in the ortho, meta, or para position.

10. The system of claim 1, wherein the UV light has a wavelength from 360-370 nm, 350-380 nm, 340-390 nm, 330-400 nm, or 315-400 nm.

11.-14. (canceled)

15. A system comprising:
(a) a composition comprising a triazene molecule according to Formula A, wherein R₁ is in the ortho, meta, or para position, wherein the first atom of R₂ and the first atom of R₃ are both an sp³ hybridized carbon; R₂=CH₂—X—CH₂—CH₂—R₃ or CH₂—CH₂—X—CH₂—R₃ wherein R₃ is C and X is CH₂, NH, S, or O; or R₃=CH₂—X—CH₂—CH₂—R₂ or CH₂—CH₂—X—CH₂—R₂ wherein R₂ is C and X is CH₂, NH, S, or O; and
(b) UV light; wherein the UV light reacts with the triazene molecule to form a diazonium species.

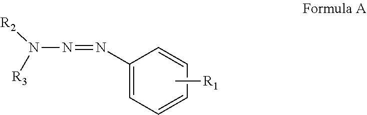

16. The system of claim 15, wherein R₁ is a carboxylic acid derivative, is an alkyne, is a bioorthogonal handle, or is a drug.

17.-19. (canceled)

20. The system of claim 15, wherein R₁ is selected from —NO₂, —CN, —CF₃, —COOH, —RCOOH, —CONHCH₃, —Br, —OMe, —H, or —CH₃.

21. The system of claim 15, wherein the composition is according to Formula B or C 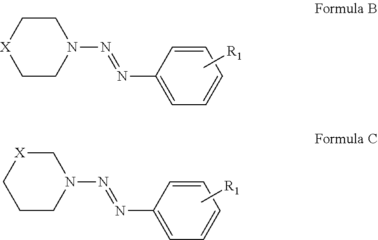
wherein X in Formula B or Formula C is CH₂, NH, S, or O.

22. (canceled)

23. The system of claim 15, wherein the UV light has a wavelength from 360-370 nm, 350-380 nm, 340-390 nm, 330-400 nm, or 315-400 nm.

24.-52. (canceled)

53. A composition comprising a triazene molecule according to Formula A, Formula B, or Formula C, wherein R₁ is in the ortho, meta, or para position, wherein in Formula A the first atom of R₂ and the first atom of R₃ are both an sp³ hybridized carbon.

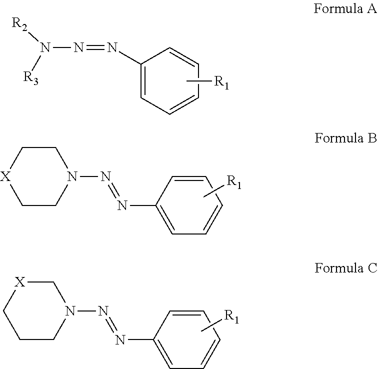

54. The composition of claim 53, wherein R₁ is a carboxylic acid derivative, an alkyne, a bioorthogonal handle, or a drug.

55.-57. (canceled)

58. The composition of claim 53, wherein R₁ is selected from —NO₂, —CN, —CF₃, —COOH, —RCOOH, —CONHCH₃, —Br, —OMe, —H, or —CH₃.

59. The composition of claim 53, wherein X in Formula B or Formula C is selected from CH₂, NH, S, or O.

60. The composition of claim 53, wherein the composition is capable of releasing a diazonium species upon exposure to UV light, wherein the UV light has a wavelength from 360-370 nm, 350-380 nm, 340-390 nm, 330-400 nm, or 315-400 nm.

61.-70. (canceled)

71. The composition of claim 53, wherein the composition is for protein modification.

72. The composition of claim 53, wherein the composition is a probe.

73. The composition of claim 53, wherein the composition comprises a detectable moiety or a tag, wherein the detectable moiety is a fluorescent moiety or a colorimetric moiety; wherein the tag is an affinity tag.

74.-76. (canceled)

77. The composition of claim 53, wherein the composition modifies aromatic nucleophiles, tyrosine residues, histidine residue, tryptophan residues, or a combination thereof.

78.-135. (canceled)
